(19) United States
(12) Patent Application Publication
(10) Pub. No.: US 2002/0022815 A1
(43) Pub. Date: Feb. 21, 2002

(54) Nursing pad

(75) Inventor: Koji Yamaji, Zentsuuji-shi (JP)

Correspondence Address: LORUSSO & LOUD, 3137 Mt. Vernon Avenue, Alexandria, VA, 22305, US

(73) Assignee: BELUSANTE CO., LTD.

(21) Appl. No.: 09/739,679

(22) Filed: Dec. 20, 2000

(30) Foreign Application Priority Data
Aug. 18, 2000 (JP) 2000-248039

(51) Int. Cl. A41C 3/08; A41C 3/00; A61F 13/15; A61F 13/20

(52) U.S. Cl. 604/385.07; 604/385.03; 604/385.05; 604/387; 450/28; 450/37

(57) ABSTRACT

A nursing pad adapted to be interposed between a user's bra and the user's breast received in the bra, including an inner, flexible, water permeable layer, an exterior, flexible, water impermeable layer having a peripheral portion bonded to the inner layer, and an absorbent layer interposed between the inner and exterior layers, wherein a slit extends from a periphery of the pad inward to form separated portions on both sides thereof so that the pat can form to the contour of the user's breast, when the separated portions are overlapped. An adhesive is provided on at least one of the separated portions for maintaining the overlap of the separated portions.

### BACKGROUND OF THE INVENTION

1. Field of the Invention

The present invention relates to a nursing pad for absorbing breast milk leaking from a lactating user'3 nipple.

2. Description of the Prior Art

This type of nursing pad is inserted into a cup of a user's undergarment (which may be hereinafter referred to as “bra”) covering the use's breast. It is desirable that the pad is of such a contoured shape as to encompass the use's breast (a generally hemispherical shape) for preventing the pad from being crinkled and getting out of position with respect to the breast while being used.

One conventional contoured nursing pad is shown in FIG. 12 and FIG. 13. The conventional nursing pad shown in FIG. 12 and FIG. 13, designated generally as A, comprises an inner layer 1 having a peripheral edge 10, an exterior layer 2 having a peripheral edge 20, and an absorbent layer 3 for absorbing excess milk, the peripheral edge 10 of the inner layer 1 and the peripheral edge 20 of the exterior layer 2 being bonded together (by heat-bonding, for instance) with the absorbent layer 3 interposed therebetween. In the nursing pad A, the inner layer 1 is a flexible, water permeable sheet such as a nonwoven fabric, while the exterior layer 2 is a laminate composed of an outer sheet 21, such as a paper or a nonwoven fabric, and a synthetic resin waterproof film 22 as shown in FIG. 13.

The nursing pad A is produced by being heated under a pressure in a mold to melt the synthetic resin waterproof film 22. Upon being cooled, the film 22 is hardened into a generally hemispherical shape as shown in FIG. 12 so that the pad A can retain its hemispherical shape.

This kind of pads are generally provided in various sizes (the diameter D in FIG. 12 is 110 mm, 120 mm, 130 mm etc.) to fit different sized breasts.

Such a heat-molded nursing pad A as shown in FIG. 12 has an advantage of fitting the user's breast well. However, since the synthetic resin waterproof film 22 of the exterior layer 2 is heat-cured, the exterior layer 2 is so hard and stiff that the pad is uncomfortable to the wearer.

Additionally, the conventional nursing pads must be provided in various sizes (diameters), generally large, medium and small, according to the needs of the users (the sizes of breasts), which means a plurality of production lines and supervisory systems are needed and thus the management systems are unavoidably complicated.

### SUMMARY OF THE INVENTION

It is, therefore, an object of the present invention to provide a nursing pad for absorbing excess milk leaking from a lactating woman's breast which is not heat-cured and thus flexible, but can be easily formed to the contour of the user's breast.

Another object of the present invention is to provide a nursing pad which is size (diameter)-adjustable and thus can be provided in one size.

In accomplishing the foregoing object, there is provided in accordance with the present invention a A nursing pad adapted for attaching to a cup of a use's bra and interposed between the cup of the bra and the user's breast received in the cup of the bra, said nursing pad comprising:

an inner, flexible, water permeable layer;

an exterior, flexible, water impermeable layer having a peripheral portion bonded to said inner layer;

an absorbent layer interposed between said inner layer and said exterior layer;

a slit extending from a periphery of said pad inward to form separated portions on both sides thereof so that said pat can form to the contour of the user's breast, when said separated portions are overlapped; and an adhesive provided on at least one of said separated portions for maintaining the overlap of said separated portions.

A nursing pad of the present invention comprises an inner layer of a flexible water permeable material, an exterior layer of a flexible water impermeable material and an absorbent layer interposed between the inner layer and the exterior layer, the inner layer and the exterior layer being bonded together along the periphery of the absorbent layer. The bonding may be by heat-bonding or with hot-melt.

The absorbent layer may be a well-known absorbent material such as an absorbent paper, a moisture hygroscopic polymer sheet, a fluffy pulp or multiple layers of any combination thereof.

The inner layer may be a flexible, soft nonwoven fabric. The exterior layer may include an outer sheet such as a paper or a nonwoven fabric and a synthetic resin waterproof film laminated thereon or may be a single-layer of a synthetic resin film (serving as a water-proof material). The size of the pad in plain configuration may be, for example, as large as about 130 mm to 140 mm in diameter.

The nursing pad of the present invention has a slit having a predetermined length and extending from the periphery of the pad inward to form separated portions on both sides thereof. The length of the slit may be, for example, 35 mm to 45 mm. The slit is not necessarily directed toward the very center of the pad but may make a small angle (less than ±150, for instance) with respect to the line connecting the edge of the slit at the periphery of the pad and the center of the pad.

The inner layer and the outer layer are also bonded together at a narrow width, 2 mm for instance, on both sides of the slit so that the absorbent material does not extend out at the slit. The slit allows the separated portions formed on both sides thereof to be overlapped one over the other at an arbitrary angle (the angle made by the edges of the slit is 30° to 90°, for instance).

An adhesive is provided on at least one of the separated portions for maintaining the overlap of the separated portions. The adhesive may be a double-faced tape with its one side bonded to the separated portion and with the other side thereof being detachably covered with a protective paper. Alternatively, the surface of the adhesive may be directly covered with a wrapping film for the nursing pad. In this case, the inner surface of the wrapping film is preferably provided with a protective coating such as silicone. The nursing pad may be wrapped after being folded in half.

The nursing pad may be used as follows. When unwrapped, the pad is flat. The user decides the extent (angle) of overlap of the separated portions on both sides of the slit according to her preference (or to the size of the breast to be covered). Then the pad is formed to the contour of the use's breast with the exterior layer side of the pad bulging outward into a convex form. At that time, as the extent of the overlap is larger, the peripheral length of the pad becomes shorter and the diameter thereof becomes smaller. For example, in the case of a pad of 130 mm in diameter in plain configuration, when the separated portions are overlapped at an angle of about 90°, the height of the center part of the pad will be as large as about 40 mm and the diameter of the pad will be about 110 mm. The height of the center part and the diameter of the pad can be adjusted by changing the extent of the overlap of the separated portions. The pad is maintained in the contoured shape by affixing the separated portions to each other with the adhesive provided thereon. Then the pad is inserted into the user's bra and affixed to the inner surface thereof.

Since the nursing pad of the present invention can be formed to the contour of the user's breast by adjusting the overlapping of the separated portions on both sides of the slit, the pad does not need to be molded under heat. Therefore, the exterior layer of the nursing pad is not heat-cured and thus feels soft in use (the periphery of the pad is heat-bonded but the region is very small).

In the above pad, one of the separated portions can contain no or a reduced amount of the absorbent material so as to become thinner than the other. In this case, the separated portions are overlapped with the thinner separated portion being placed outward. The other separated portion contains as thick absorbent material as the remaining region of the pad and can absorb excess milk.

In the case where one of the separated portions is thinner than the other, when the separated portions are overlapped, the thickness of the overlapped part can be reduced as compare with the case where each of the separated portions contains the absorbent material. Namely, in the case where both separated portions contains as thick absorbent material as the remaining region of the pad, the overlapped part is twice as thick as the remaining region. However, when one of the separated portions is a thinner than the other, the thickness of the overlapped part is smaller than twice the thickness of the remaining region (increase in thickness can be reduced). When the thickness of the overlapped part of the separated portions is reduced, the user's clothing worn over the nursing pad is not (or less) partially protruded and bulkiness given by the overlapped part in use can be eliminated or decreased.

In another aspect of the present invention, there is provided a nursing pad adapted for attaching to a cup of a use's bra and interposed between the cup of the bra and the user's breast received in the cup of the bra, said nursing pad comprising:

an inner, flexible, water permeable layer;

an exterior, flexible, water impermeable layer having a peripheral portion bonded to said inner layer;

an absorbent layer interposed between said inner layer and said exterior layer;

a row of perforations extending from a periphery of said pad inward to form on both sides thereof two separable portions which, when the pad is torn along said perforations, can be separated from each other such that the separated portions can be overlapped to form to the contour of the user's breast, and

an adhesive provided on at least one of said separable portions for maintaining the overlap of said separated portions.

In the above nursing pad, tearing the pad along the perforations can form a slit which is similar to that of the first embodiment. Before being torn, the separable portions are continuous and are not warped individually so that the pad is easy to handle in manufacturing and wrapping as compared with the pad having separated portions already separated by a slit. One of the separable portions may be absorbent material-free or may contain a less thick absorbent material so as to become thinner than the other.

### BRIEF DESCRIPTION OF THE DRAWINGS

Other objects, features and advantages of the present invention will become apparent from the detailed description of the preferred embodiments which follow, when considered in light of the accompanying drawings, in which:

FIG. 1 is a plan view of a nursing pad of a first embodiment of the present invention as seen from the exterior layer side thereof;

FIG. 2 is an enlarged cross-sectional view taken from the line II-II of FIG. 1;

FIG. 3 is a plan view of nursing pad illustrating a state in use;

FIG. 4 is an enlarged cross-sectional view taken from the line IV-IV of FIG. 3;

FIG. 5 is a plan view of a nursing pad of a second embodiment of the present invention as seen from the exterior layer side thereof;

FIG. 6 is an enlarged cross-sectional view taken from the line VI-VI of FIG. 5;

FIG. 7 is an enlarged cross-sectional view taken from the line VII-VII of FIG. 5;

FIG. 8 is an enlarged cross-sectional view of a nursing pad of FIG. 5, equivalent to FIG. 4;

FIG. 9 is a plan view of a nursing pad of a third embodiment of the present invention as seen from the exterior layer side thereof;

FIG. 10 is a plan view of a nursing pad of a fourth embodiment of the present invention as seen from the exterior layer side thereof;

FIG. 11 is a plan view of a nursing pad of a fifth embodiment of the present invention as seen from the exterior layer side thereof;

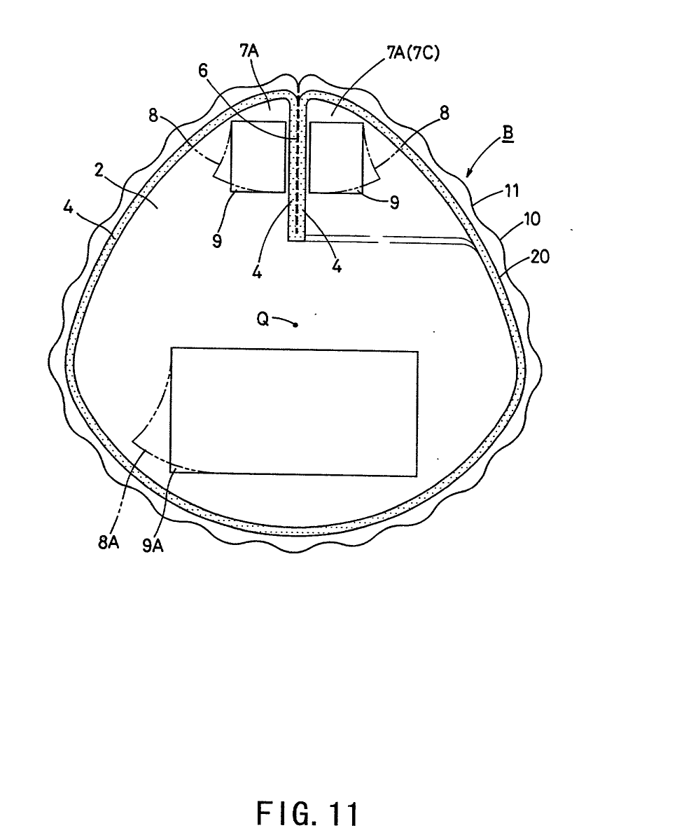

FIG. 12 is a cross-sectional view of a conventional nursing pad; and

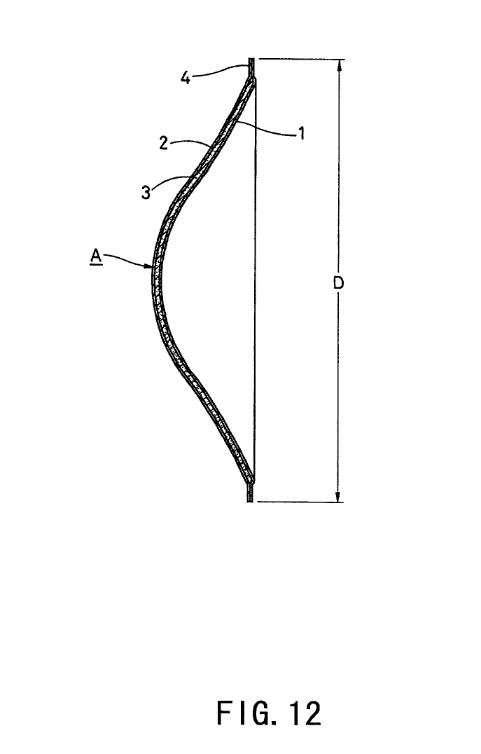

FIG. 13 is a enlarged cross-sectional view of a portion of FIG. 12.

### DETAILED DESCRIPTION OF THE PREFERRED EMBODIMENTS OF THE INVENTION

First Embodiment

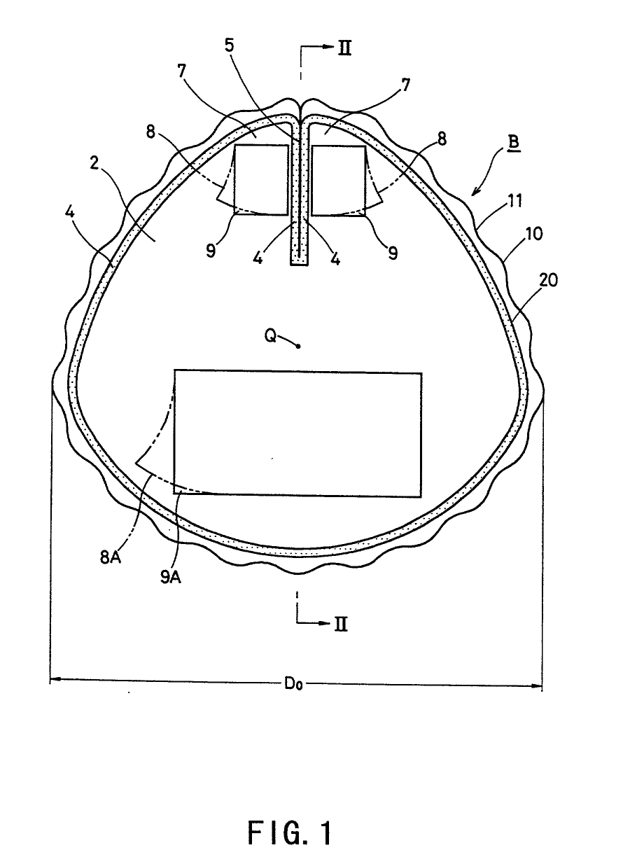

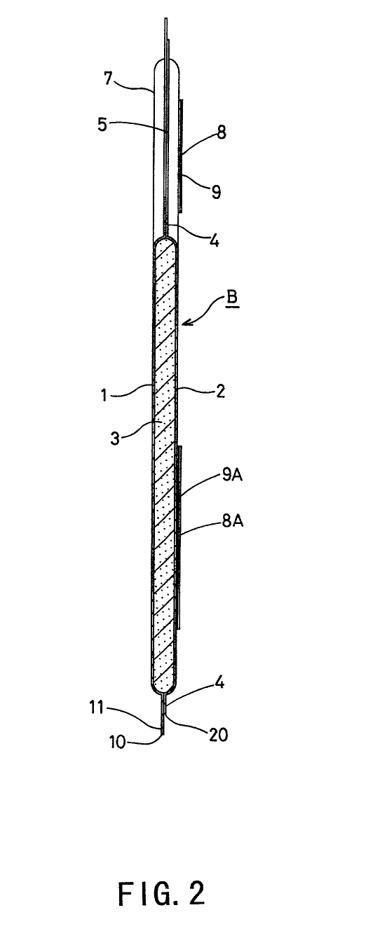

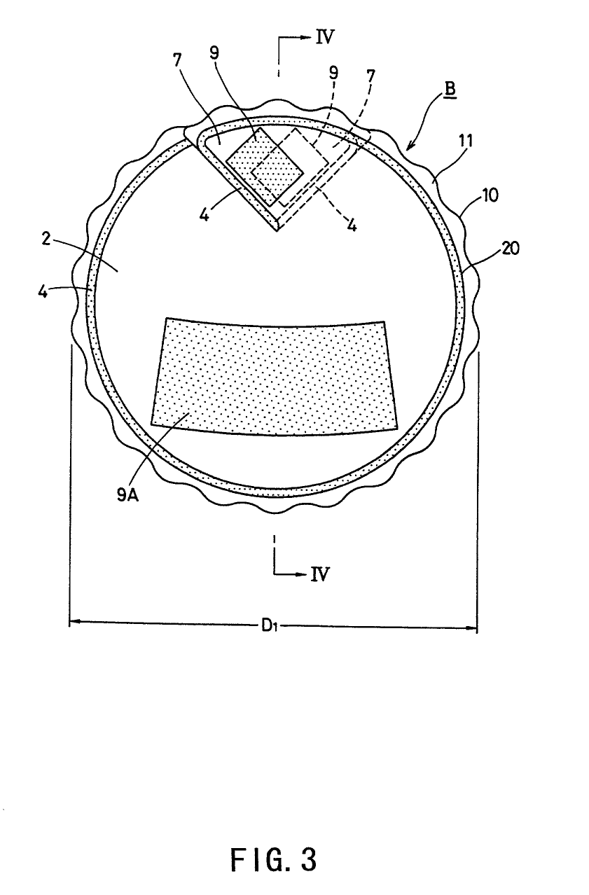

As shown in FIG. 1 and FIG. 2, a nursing pad, designated generally as B, of a first embodiment according to the present invention comprises an inner layer 1 of a water permeable material, an exterior layer 2 of a water impermeable material, and an absorbent layer 3 interposed between the inner layer 1 and the exterior layer 2. The inner layer 1 and the exterior layer 2 are bonded together along the periphery of the absorbent layer 3. In this embodiment, the pad B has a generally round triangle shape in plan configuration as shown in FIG. 1 and becomes closer to a complete circle when, as described later, separated portions 7 and 7 on both sides of a slit 5 are overlapped as shown in FIG. 3.

The absorbent layer 3 may be a well-known absorbent material such as an absorbent paper, a moisture hygroscopic polymer sheet, a fluffy pulp or a composite or laminate of any combination thereof.

The inner layer 1 is a flexible, soft nonwoven fabric which is water permeable and thus allows the excess milk leaking from a user's nipple to wick through it to the absorbent layer 3.

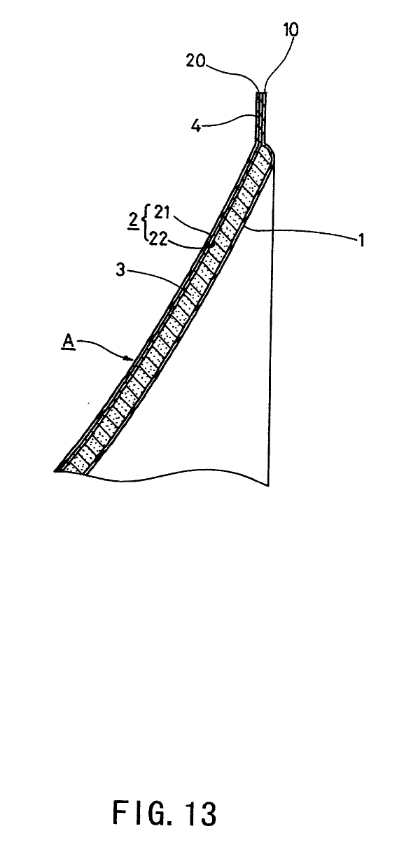

The exterior layer 2 is a flexible, synthetic resin film. The exterior layer 2 may be a well-known type such as a laminate of an outer sheet 21 (a paper or a nonwoven fabric sheet) and a synthetic resin waterproof film 22 as shown in FIG. 13. The outer sheet 21 and the film 22 may be bonded to each other to form a unitary sheet or may be separated from each other.

In the nursing pad B of the first embodiment, the inner layer 1 and the exterior layer 2 are bonded together to form a bonded part 4 having a narrow width (2 mm to 3 mm, for instance) and extending in a loop along the periphery of the absorbent layer 3 with the absorbent layer 3 being interposed therebetween. The bonding between the inner and exterior layers 1 and 2 may be by heat-bonding or by hot melt.

The inner layer 1 and the exterior layer 2, which are illustrated as rather thick in each of the cross-sectional views for the sake of convenience, are constituted of very thin material in reality. The thickness of the absorbent material of the absorbent layer 3 is suitably about 3 mm to 4 mm but not limited. In this first embodiment, the thickness of the absorbent material is substantially uniform throughout the pad including the separated portions 7 and 7.

In the nursing pad B of the first embodiment, the inner layer 1 is larger in size than the exterior layer 2 so that the periphery 10 of the inner layer 1 is extended outward around the periphery 20 of the exterior layer 2. Namely, in the nursing pad B, the periphery 10 of the inner layer 1 is extended outward around the bonded portion 4 at a predetermined width (4 mm to 5 mm, for instance). The extended portion, designated as 11, of the inner layer 1 around the periphery 20 of the exterior layer 2 is constituted of a single sheet of nonwoven fabric and thus maintains flexibility. The extended portion 11 of the inner layer 1 is provided with embossed patterns of waves or flower leaves thereon for decoration, if desired. The extended portion 11 of the inner layer 1 prevents edges of the bonded portion 4 from directly contacting with the user's skin so that possible skin irritation where the bonded portion 4 contacts the user's breast may be prevented.

The nursing pad B of the first embodiment shown in FIG. 1 and FIG. 2 has a slit 5 having a predetermined length (for example, about 35 mm to 45 mm) and extending toward the center Q (see FIG. 1) of the nursing pad B. The inner layer 1 and the exterior layer 2 are also bonded together on both sides of the slit 5 at a narrow width, 2 mm for instance. Therefore, the bonded portion 4 is continuous throughout the periphery of the pad so that the absorbent material of the absorbent layer 3 does not extend out from between the inner later 1 and the exterior layer 2.

On both sides of the slit 5 are formed separated portions 7 and 7, which can be overlapped at an arbitrary angle (the angle formed by the edges of the slit is, for example, between 30° to 90°).

Each of the separated portions 7 and 7 is provided with adhesives 9 and 9 for maintaining the overlap of the separated portions 7 and 7. Designated as 8 is a protective paper covering each of the adhesive surfaces and being peeled away in using the pad. The protective papers 8 and 8 may be substituted by one large paper covering both the adhesive 9 and 9. In another embodiment, adhesive may be provided on only one of the separated portions 7 and 7.

Additionally, the nursing pad of the first embodiment is provided with an adhesive 9A with a protective paper 8A in the lower part on the outer surface of the exterior layer 2. The adhesive 9A is for affixing the pad to the inner surface of the use's bra to hold the pad in place. In this embodiment, one of the adhesives 9 and 9 on the separated portions 7 and 7 which is not used to maintain the overlap of the separated portions 7 and 7 is also used to affix the nursing pad to the inner surface of the user's bra. In another embodiment, the protective papers 8, 8 and 8A covering the adhesives 9, 9 and 9A may be substituted for the wrapping film of the nursing pad (having a protective property on the inner surface thereof).

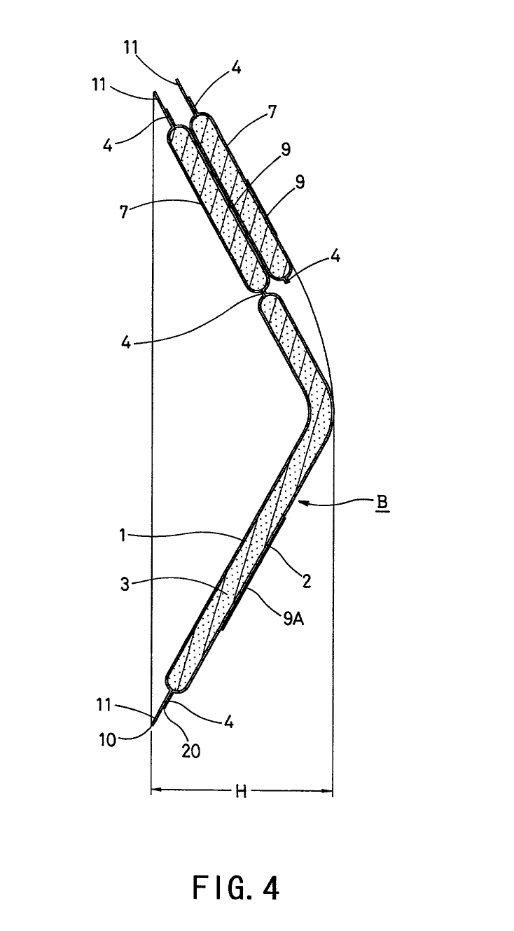

The nursing pad B of the first embodiment is used as follows. When unwrapped, the nursing pad is flat as shown in FIG. 1 and FIG. 2. In the case where the pad is wrapped folded in half during storage and transportation, it becomes flat when unwrapped and unfold. One of the protective papers 8 and 8 is peeled off to expose the adhesive 9 thereunder, the separated portions 7 and 7 are overlapped at a certain desired angle (the angle formed by the edges of the slit is 90° in FIG. 3) as shown in FIG. 3 and FIG. 4 and affixed to each other with the adhesive 9, whereby the nursing pad B is maintained in a generally cup shape with the exterior layer 2 side bulged outward as shown in FIG. 3 and FIG. 4. At this time, the diameter D1 of the nursing pad B become smaller according to the overlapping angle of the separated portions 7 and 7. For example, in the case where the pad is as large as about 130 mm to 140 mm in diameter D₀ in the plain configuration shown in FIG. 1 and the separated portions 7 and 7 are overlapped at an angle of about 90°, the diameter of the pad B becomes as small as about 110 mm to 115 mm (D₁) and the height (H in FIG. 4) of the center part of the pad becomes as large as about 40 mm. The height H of the center part of the pad and the diameter of the pad (D₀ to D₁) can be adjusted by changing the overlapping angle of the separated portions 7 and 7.

After being formed to the contour of the user's breast as above, the other protective paper 8 on the other separated portion and the protective paper 8A of the adhesive 9A are peeled off and the pad B is affixed to the inner surface of the user's bra with the adhesive 9 and 9A.

The nursing pad B of the first embodiment, which can be formed to the contour of the user's breast only by overlapping the separated portions 7 and 7, does not need heat-molding to form into a contoured shape required in conventional nursing pads. Therefore, in the nursing pad B of the embodiment, since the exterior layer 2 is not heat-cured, the pad does not feel stiff but soft in use.

Second Embodiment

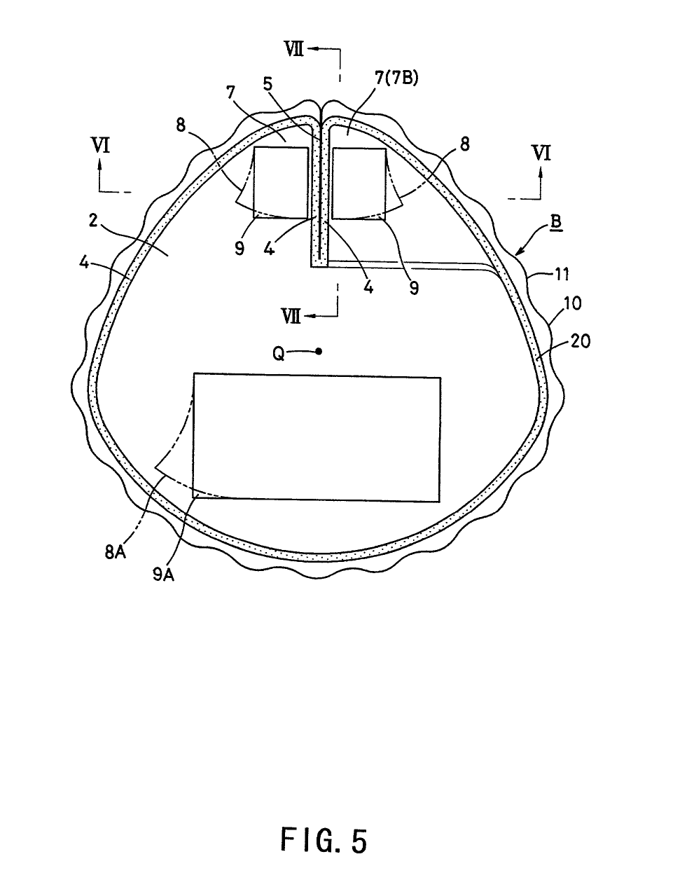

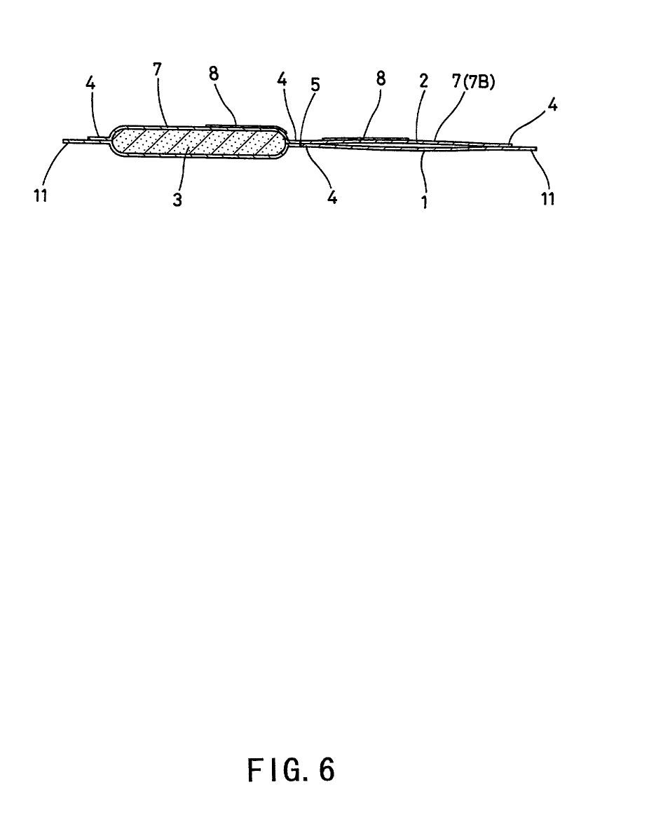

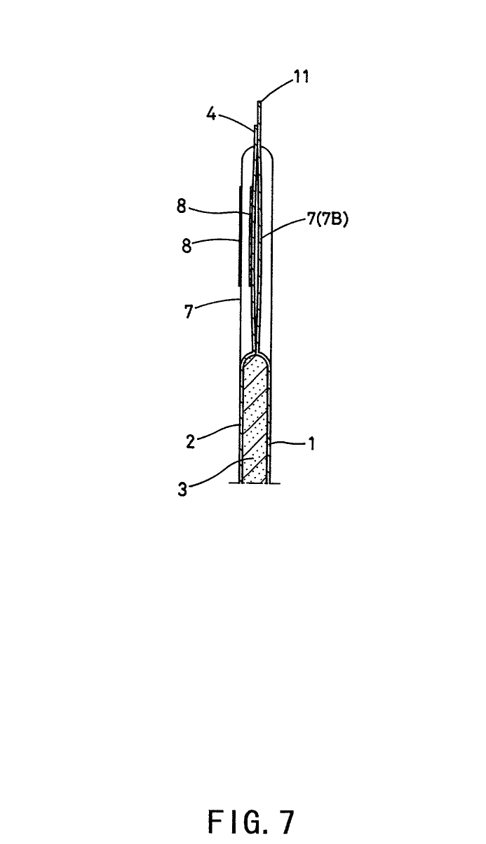

A nursing pad of a second embodiment shown in FIG. 5 to FIG. 8 is a modification of the first embodiment. In the nursing pad B of the second embodiment, one of the separated portions 7 and 7 formed by the slit 5 (the right portion in FIG. 5) and adapted to be overlapped one over the other contains no absorbent material and is thinner than the other. As shown in FIG. 6 to FIG. 8, the thinner separated portion, designated as 7B contains no absorbent material and is constituted of only the inner layer 1 and the exterior layer 2. The other separated portion 7 (the left in FIG. 5) contains as thick absorbent material (about 3 mm to 4 mm for instance) as the remaining region so as to absorb excess milk.

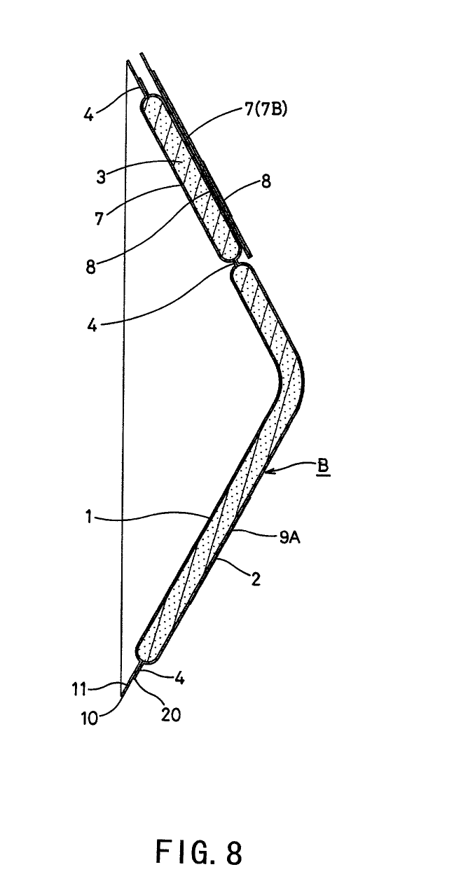

When the nursing pad B of the second embodiment is used, the separated portions are overlapped with the thin separated portion 7B being placed outward as shown in FIG. 8 as is the case with the first embodiment. In the case where one of the separated portions is thinner than the other, when the separated portions are overlapped, the increase in thickness of the overlapped part can be reduced as compared with the first embodiment. Namely, in the case where both separated portions contain as thick absorbent material as the remaining region, the overlapped part is twice as thick as the remaining region (7 mm to 8 mm) as shown in FIG. 4. However, when one of the separated portions is thinner than the other as shown FIG. 8, the overlapped part in only slightly thicker than the remaining region (increase in thickness can be reduced). When the increase in thickness of the overlapped part of the separated portions is reduced, the user's clothing worn over the nursing pad is not (or less) partially protruded and bulkiness given by the overlapped part of the separated portions in use can be eliminated or decreased.

In the second embodiment shown in FIG. 5 to FIG. 8, the thinner separated portion 7B contains no absorbent material. In this case, the increase in thickness of the overlapped part of the separated portions 7 and 7B can be minimized. However, it may be difficult for the thinner separated portion to maintain the shape and thus the pad may be difficult to handle. Thus, in another embodiment, the thinner separated portion 7B may contain a thinner absorbent material than the remaining region of the pad (half as thick absorbent material as the remaining region, for instance).

Third Embodiment

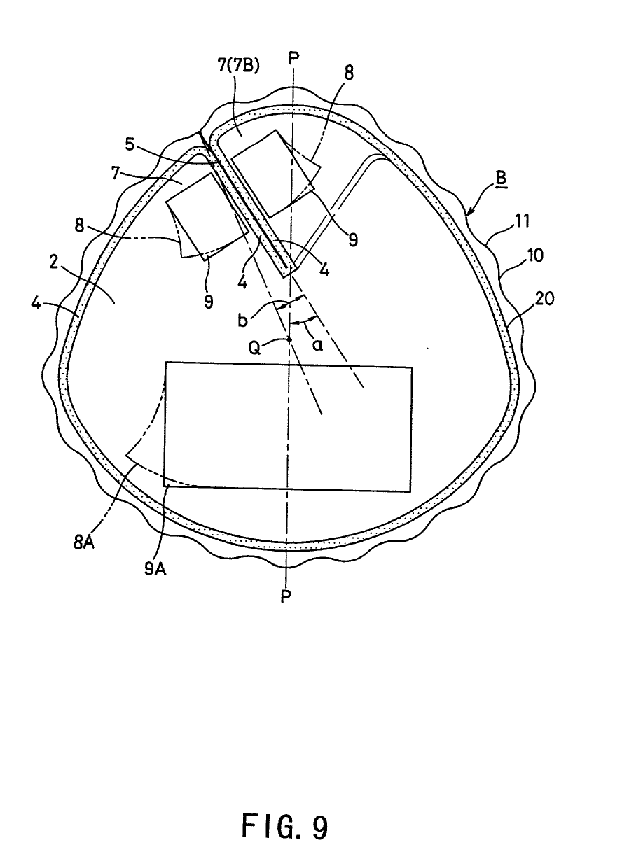

A nursing pad B of a third embodiment shown in FIG. 9 is a modification of the second embodiment. In the third embodiment, the nursing pad has a slit 5 extending from a point slightly offset circumferentially from the top of the pad at a given angle “a” (for example about 30° in the example in FIG. 9) with respect to the line P-P passing through the top of the pad and the center Q thereof, and inclined at a small angle “b” (for example about 10° in the example in FIG. 9) toward the center of the pad. In this embodiment, too, one of the separated portions is thinner than the other and the separated portion are overlapped with the thinner separated portion placed outward to form to the contour of the user's breast.

Fourth Embodiment

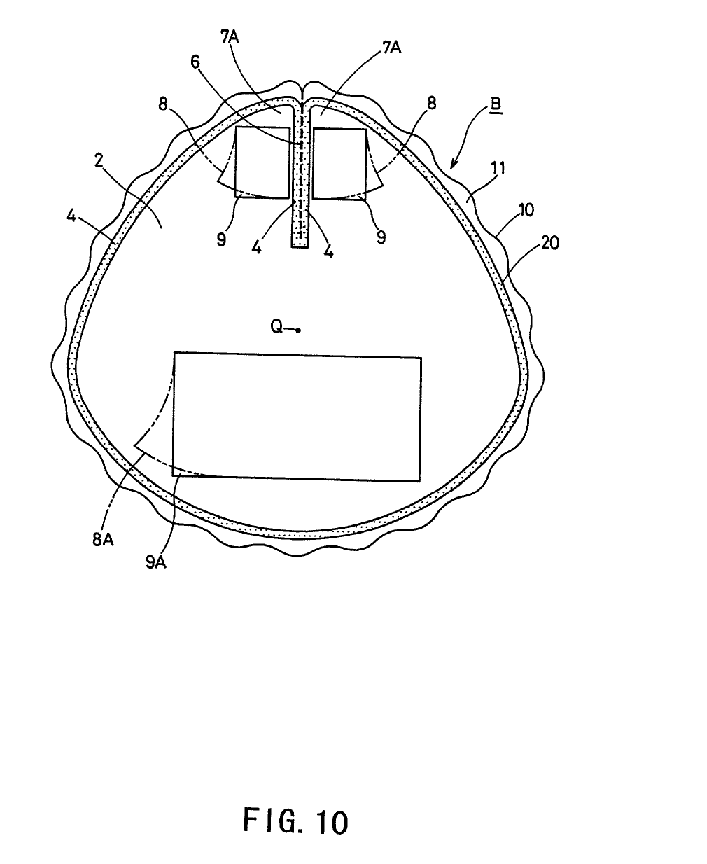

A nursing pad B of a fourth embodiment shown in FIG. 10 has a row of perforations 6 which form on both sides thereof two separable portions 7. Other structures are the same as the first embodiment.

In the nursing pad B of the fourth embodiment, tearing the pad along the perforations 6 can form the slit 5 of the first embodiment. Since the separable portions 7 and 7 are not separated before use, each of the separable portions is not warped individually before use. Therefore, the pad is easy to handle in manufacturing and wrapping as compared to the pad of the first embodiment in which portions 7 and 7 are already separated by a slit before packaging.

Fifth Embodiment

A nursing pad B of a fifth embodiment shown in FIG. 8 is a modification of the fourth embodiment. In the fifth embodiment, one of the separable portions (the right in FIG. 8) is thinner than the other as is the case with the second embodiment.

In the nursing pad B of the fifth embodiment, tearing the pad along the perforations 6 can form the slit 5 of the first embodiment as is the case with the forth embodiment, and the pad can be formed to the contour of the user's breast shown in FIG. 8 by overlapping the separated portions with the thinner separated portion (designated as 7C) placed outward (serving the same function as that of the second embodiment).

In further embodiments of the present invention, the biased slit of the third embodiment may be applied to the first, second, fourth and fifth embodiments.

The nursing pad of the present invention has good effects as follows.

Effect of the First Embodiment

The nursing pad B of the first embodiment of the present invention has a slit 5 having a predetermined length and extending from the periphery of the pad inward to form separated portions on both sides thereof such that the separated portions can be overlapped to form to the contour of the user's breast and that the size (diameter) of the pad can be adjusted at will.

The nursing pad B can be formed to the contour of the user's breast only by overlapping the separated portions 7 and 7.

The nursing pad B does not need heat-molding required in conventional nursing pads. Since the exterior layer 2 is not heat-cured (maintains flexibility), the pad feels not still but soft in use.

The nursing pad B can be adjusted in size according to the user's preference (the size of the breast) only by changing the overlapping angle of the separated portions. Therefore, it is not necessary to provide various sized pads so that the production line and the supervising system thereof can be simplified.

Effect of the Second and fifth Embodiments

In the nursing pad B of the second and fifth embodiments of the present invention, one of the separated portions contains no or less thick absorbent material and is thinner than the other. When the separated portions are overlapped, the thickness of the overlapped part is thinner than the twice the thickness of the remaining region of the pad (increase in thickness can be reduced).

Therefore, the nursing pad of the second and fifth embodiments has an effect, in addition to the effects of the first embodiment, that the user's cloth worn over the pad is no or less protuberant (not conspicuous) and bulkiness given by the overlapped part can be eliminated or decreased.

Effect of the fourth Embodiment

The nursing pad of the fourth embodiment of the present invention has a row of perforations having a predetermined length and extending from a periphery of the nursing pad inward in place of the slit of the first embodiment. Tearing the pad along the perforation can form the slit of the first embodiment but the separable portions are continuous before being torn.

In the nursing pad of the fourth embodiment, each of the separable portions is not warped individually so that it is easy to handle in manufacturing and wrapping.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all the changes which come within the meaning and range of equivalency of the claims are therefore intended to be embraced therein.

The teachings of Japanese Patent Application No. 2000-248039, filed Aug. 18, 2000, inclusive of the specification, claims and drawings, are hereby incorporated by reference herein.

What is claimed is:

1. A nursing pad adapted to be interposed between a user's bra and the user's breast received in the bra, said nursing pad comprising:
an inner, flexible, water permeable layer;
an exterior, flexible, water impermeable layer having a peripheral portion bonded to said inner layer;
an absorbent layer interposed between said inner layer and said exterior layer;
a slit extending from a periphery of said pad inward to form separated portions on both sides thereof so that said pat can form to the contour of the user's breast, when said separated portions are overlapped; and
an adhesive provided on at least one of said separated portions for maintaining the overlap of said separated portions.

2. A nursing pad as recited in claim 1, wherein one of said separated portions is thinner than the other.

3. A nursing pad adapted to be interposed between a user's bra and the user's breast received in the bra, said nursing pad comprising:
an inner, flexible, water permeable layer;
an exterior, flexible, water impermeable layer having a peripheral portion bonded to said inner layer;
an absorbent layer interposed between said inner layer and said exterior layer;
a row of perforations extending from a periphery of said pad inward to form on both sides thereof two separable portions which, when the pad is torn along said perforations, can be separated from each other such that the separated portions can be overlapped to form to the contour of the user's breast, and
an adhesive provided on at least one of said separable portions for maintaining the overlap of said separated portions.

4. A nursing pad as recited in claim 3, wherein one of said separable portions is thinner than the other.
